(19) United States
(12) Patent Application Publication
(10) Pub. No.: US 2003/0105512 A1
(43) Pub. Date: Jun. 5, 2003

(54) Stent containing medically treating material

(76) Inventor: Nozomu Kanesaka, Old Tappan, NJ (US)

Correspondence Address: KANESAKA AND TAKEUCHI, 1423 Powhatan Street, Alexandria, VA, 22314, US

(21) Appl. No.: 10/000,810

(22) Filed: Dec. 4, 2001

(51) Int. Cl. A61F 2/06

(52) U.S. Cl. 623/1.15

(57) ABSTRACT

A stent is formed of a body member arranged in a cylindrical shape for forming the stent to be placed in a body lumen, at least one hollow portion with at least one open surface formed in the body member, and a medically treating material disposed in the hollow portion. The medically treating material held in the hollow portion is gradually released through the open surface to treat a portion where the stent is disposed for a desired term.

### BACKGROUND OF THE INVENTION AND RELATED ART STATEMENT

The invention relates to a flexible stent with a medically treating material, i.e. drug, to be implanted in body lumen, such as an artery, to maintain patency thereof.

Coronary artery disease is the leading cause of death in the Western world. Atherosclerosis is a focal, intimal disease relating to large and medium-sized vessels. The typical atherosclerotic lesions are called plaques which reduce the inside of the arterial vessels by occlusion. PTCA balloon procedure has been successfully used for the treatment of coronary artery stenosis by mechanically opening the vessel diameter. However, a significant number of patients is required to have a further treatment due to various reasons including dissections, thrombus, and restenosis on the same lesion. The concept of endovascular stents was that they would act as a scaffold to tack down dissection flaps, limit elastic recoil, and possibly reduce the incidence of restenosis.

There have been introduced various types of stents. From a viewpoint of methods for expanding the stent, the stents can be categorized as a self-expandable stent which can expand by itself, and a balloon expandable stent which is expanded by a balloon catheter. From a viewpoint of materials, the stents can be categorized into a tubular stent and a wire stent, and from a viewpoint of methods for manufacturing the stent, the stents can be categorized as an etched stent and a laser cut stent. In all the types of stents, the stent expands from an initial diameter to a larger diameter so as to be suitable for a particular size of the body cavity.

The stent is very effective in supporting the blood vessel, but the stent and/or delivery of the stent may hurt the blood vessel wall because the stent is delivered through a meandering wall vessel by the catheter. In this case, it is desirable to provide a drug for the portion of the injured blood vessel wall. On the other hand, the blood vessel wall or a portion near the blood vessel has a problem or is hurt by disease.

Therefore, recently, the stent is processed to have a coating containing a drug for treatment of the blood vessel wall or a portion near the blood vessel. For example, the coating of the stent is disclosed in U.S. Pat. No. 6,153,252, W00010552, and EP0691130.

The coating is effective for treatment of the blood vessel wall and the area adjacent thereto, but the coating contacts the tissue or blood all the time when the stent is implanted. And the maximum coating thickness is generally limited to 10 to 50 microns. Therefore, the effective time or duration by the drug release is limited. Also, the coating may peel off from the stent in the area where bending takes place when the stent is expanded in use.

Accordingly, an object of the invention is to provide a stent, which contains a drug or a drug matrix for treatment and can release the drug slowly for a long time.

Another object of the invention is to provide a stent as stated above, wherein the drug can be securely held in the stent without peeling off.

Further objects and advantages of the invention will be apparent from the following description of the invention.

### SUMMARY OF THE INVENTION

A stent of the invention is formed by a body member arranged in a cylindrical shape for forming the stent to be placed in a body lumen, at least one hollow portion with at least one open surface formed in the body member, and a medically treating material, i.e. drug or drug matrix, disposed in the at least one hollow portion. The medically treating material held in the hollow portion is gradually released through the at least one open surface to treat a portion where the stent is disposed for a desired term.

In the invention, since the drug for treatment can be disposed in the hollow portion of the body member forming the stent, a large amount of drug can be kept in the stent. Also, since the drug is mostly kept in the hollow portion, the drug can be released slowly through the open surface, as desired.

Namely, if the drug is coated on the stent, the amount of the drug coated on the stent is limited due to the thickness of the coating (generally 10 to 50 microns), but in the invention, a large amount of drug can be kept in the hollow portion forming nearly the thickness of the stent, i.e. about 1000 microns. Also, if the drug is coated, when the stent is enlarged or expanded, the drug coated on the stent may be peeled off. In the invention, since the drug is held in the non-bending hollow portion, the peeling or bending of the drug does not occur.

In the invention, the at least one hollow portion may be at least one groove formed on one of inner and outer surfaces of the body member. Namely, the one groove may be a continuous groove formed in a substantial area of the body member.

The body member may include a plurality of elongated members and a plurality of connecting members situated between the elongated members for connection. The elongated members and connecting members are arranged such that when the stent is enlarged, the elongated members are at least bent at portions near the connecting members. In this case, the at least one groove may be a plurality of elongated grooves formed at the elongated members. Since the grooves are not formed at the connecting members or the end of the elongated members where the stent bends when enlarged, the drug in the grooves can be securely held therein without peeling.

The at least one hollow portion may be a plurality of holes penetrating the body member in the direction perpendicular to the longitudinal direction of the body member. In this case, the drug can be securely held in the holes. In case the body member includes a plurality of elongated members and a plurality of connecting members situated between the elongated members for connection, as in the former case, the plurality of holes may be formed only in the elongated members.

The at least one hollow portion with at least one open surface may be formed of a central hole passing inside the body member along the longitudinal direction thereof, and a plurality of outer holes communicating between the central hole and an outside. Namely, the at least one hollow portion may be a cannula forming a wire stent.

### BRIEF DESCRIPTION OF THE DRAWINGS

FIG. 1 is a side view of a stent according to a first embodiment of the invention;

FIG. 2 is an enlarged side view of a part of the stent shown in FIG. 1;

FIG. 3 is an enlarged sectional view of the stent taken along line 3-3 in FIG. 2;

FIG. 4 is a sectional view, similar to FIG. 3, of a modified example of the stent shown in FIG. 1;

FIGS. 5 and 6 are sectional views, similar to FIG. 3, of modified examples of the stent shown in FIG. 1;

FIG. 7 is a side view of the stent according to a second embodiment of the invention;

FIG. 8 is a side view of the stent according to a third embodiment of the invention;

FIG. 9 is a side view of the stent according to a fourth embodiment of the invention;

FIG. 10 is an enlarged sectional view taken along line 10-10 in FIG. 9; and

FIG. 11 is an enlarged sectional view, similar to FIG. 10, of a modified example of the stent shown in FIG. 9.

### DETAILED DESCRIPTION OF THE PREFERRED EMBODIMENTS

Hereinafter, embodiments of the present invention will be explained with reference to the attached drawings.

A stent 10 of a first embodiment of the invention is generally shown in FIG. 1. The stent 10 has a structure similar to the stent as disclosed in U.S. Pat. No. 5,776,183. Namely, the stent 10 is formed by inner sections 11, and outer sections 12, which are arranged cylindrically. The inner section 11 includes diagonal portions 13, 14 and connecting portions 15 for connecting the diagonal portions. Each diagonal portion includes long elongated members 16, short elongated members 17, and connecting members 18. The diagonal portions 13, 14 are arranged symmetrically relative to the connecting portions 15. The inner section 11 is connected to the adjacent inner section 11 or outer section 12 by connecting members 19. The outer section 12 includes parallel members 20 and connecting members 21, and is connected to the adjacent inner section 11 by the connecting members 19.

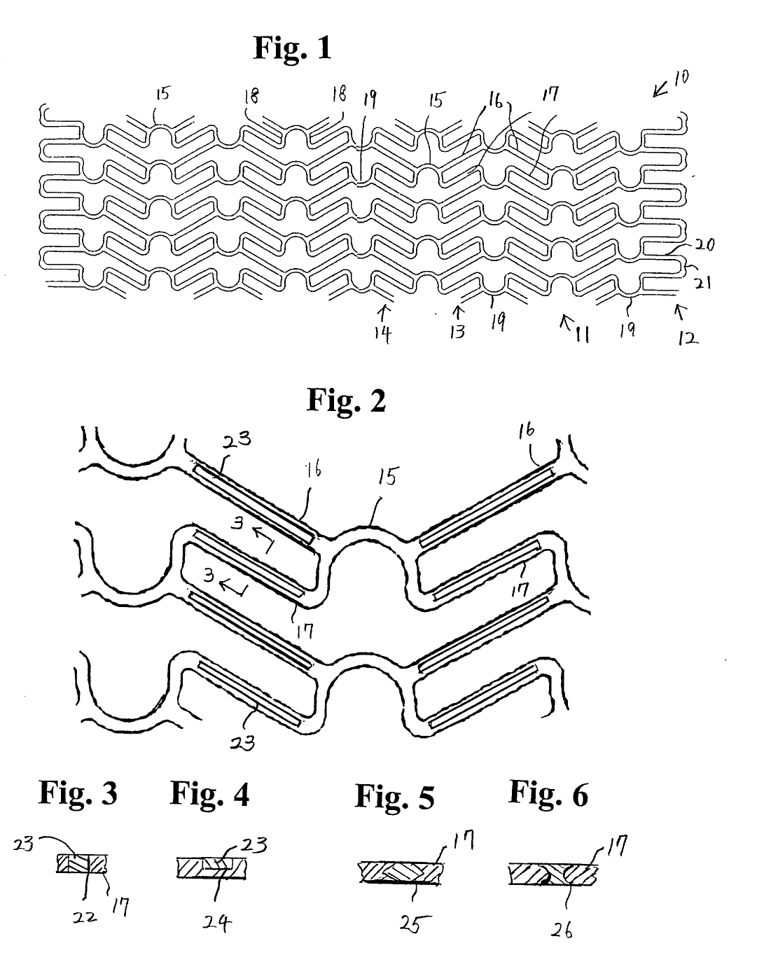

In the stent of the invention, as clearly shown in FIGS. 2 and 3, the long and short members 16, 17 have elongated holes 22 penetrating through the entire thickness of the members 16, 17. In the elongated holes 22, a drug 23 is filled.

The drug 23 filled in the elongated holes 22 may be any kind of drug, therapeutic agents, drug matrix, mixture of drugs, or other materials useful for curing or treating a portion where the stent is placed, or areas near the portion where the stent is placed. Namely, the drug includes coating materials, polymers, pharmaceutic agents, anti-angiogenic agents, and so on, including cytostatic and cytotoxic drugs and materials, such as disclosed in U.S. Pat. No. 6,153,252, WO 0,010,552, and EP 0,691,130. There is no particular limitations for the drug.

The drugs 23 may be directly filled in the elongated holes 22, or after providing a primer and/or polymer coating, such as polyurethane or PTFE, on a metal portion of the stent. When the primer or polymer coating is used, the drug can be firmly retained in the elongated holes 22. The top layer can be applied to encapsulate the drug to control the drug release rate.

The stent 10 and the elongated holes 22 may be formed by chemical etching or laser cut by using a cylindrical metal plate, such as stainless steel or nitinol. The elongated holes 22 may be formed at the same time of forming the stent 10, or formed separately.

In the invention, the elongated holes 22 are formed at portions slightly away from portions where the long and short members 16, 17 are bent when the stent is enlarged or expanded. Therefore, no high stress is applied to the drug 23 filled in the elongated holes 22 when the stent is expanded, so that the drug 23 filled in the elongated holes 22 is not peeled or separated from the stent 10. The drug 23 can be surely retained in the elongated holes 22.

When the stent 10 with the drug 23 is used, the stent 10 is mounted on a balloon catheter (not shown) as in the conventional method, and is delivered to the desired location in the blood vessel. Then, the stent 10 is expanded to be retained thereat, and the balloon catheter is removed.

In the invention, since the drug 23 is retained in the stent 10, the area on and around the portion where the stent is placed can be treated by the drug 23. The drug 23 filled in the stent 10 is selected according to the specific purpose, such as preventing undesirable response to injury of blood vessel and treatment of the blood vessel or tissue outside the blood vessel. The stent with the drug may be placed into the blood vessel for treatment of a disease near the blood vessel.

In the invention, since the drug 23 is filled in the elongated holes 22, a large amount of drug can be retained in the stent 10. The effect of the drug will last for a long time, e.g. more than one year. Also, in case the drug is deposited in the elongated holes 22 in the form of layer, the drug releasing late can be controlled. In the invention, the bending strength of the stent is kept as it is while providing the effect by the drug.

FIGS. 4-6 are modified examples of the first embodiment of the invention. In FIG. 4, instead of the elongated holes 22, elongated grooves 24 are formed, and the drug 23 is filled in the grooves 24. In this case, the strength of the long and short members 16, 17 can be reinforced. The grooves may be formed by laser cutting or etching.

In FIG. 5, a hole 25 formed in each of the long and short members 16, 17 has a shape where an inside is widened. This shape is formed by etching the stent while covering resisting layers on the outer surfaces of the stent except for the portion of the hole 25. In this case, a large amount of drug can be kept inside the hole 25. Also, in case the sizes of the outlets of the hole 25 are selected, the releasing rate of the drug can be controlled as well.

In FIG. 6, a hole 26 in each of the long and short members 16, 17 has a shape such that the outlets are formed widely. This shape is formed by etching the stent without covering by resisting layers, or by etching the stent by covering the resisting layers while controlling an etching liquid and speed. In this shape, the drug can be released quickly at an early stage of placing the stent.

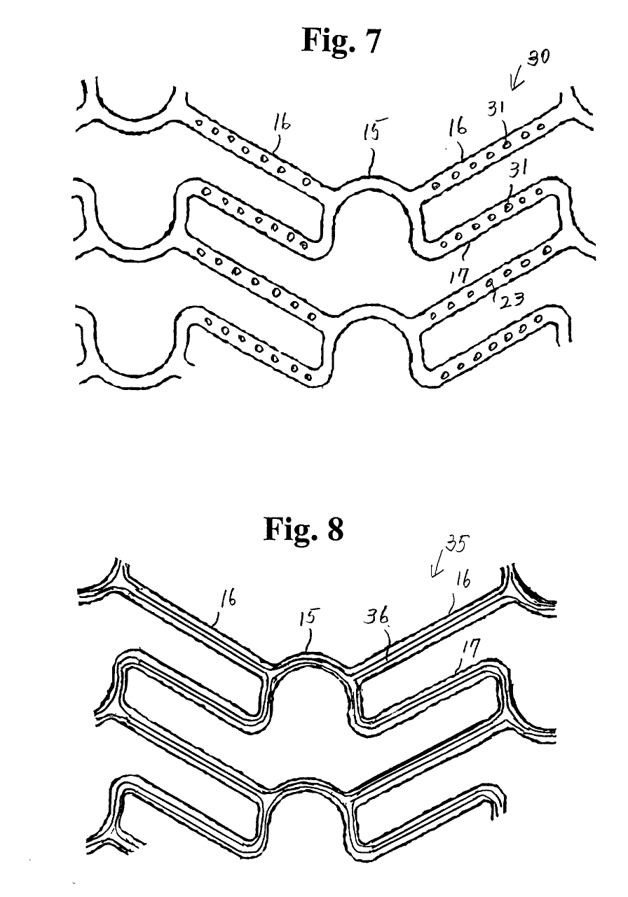

FIG. 7 shows a second embodiment of the stent 30 of the invention, which has the basic structure as in the stent 10. In the stent 30, however, instead of the elongated holes 22, a plurality of through holes 31 is formed at the long and short members 16, 17, and the drug 23 is filled in the through holes 31. The through holes 31 are located at portions slightly away from the bending portions of the long and short members 16, 17. The stent 30 operates as in the first embodiment.

FIG. 8 is a third embodiment of a stent 35 of the invention, which has the basic structure as in the stent 10. In the stent 35, however, instead of the elongated holes 22, a continuous groove 36 as shown in the modified example of FIG. 4 is formed in the members 15-21, and the drug 23 is filled in the grooves 36. The stent 35 operates as in the first embodiment.

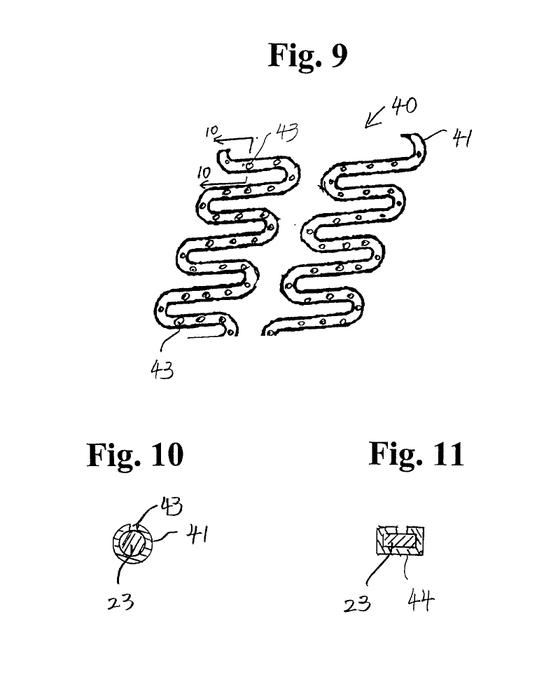

FIGS. 9 and 10 show a fourth embodiment of a stent 40 of the invention, which is a wire stent and is formed of a continuous wire 41, i.e. cannula with an inner hole, arranged obliquely in a cylindrical form. As shown in FIG. 10, the wire 41 has an inner hole 42 extending throughout an entire length thereof, and outer holes or outlets 43 passing through the wall of the wire 41 to communicate the inner hole 42 with an outside. The ends of the wire 41 are closed by caps or other suitable means. The drug 23 is filled in the inner hole 42, and is released through the outlets 43 when used.

FIG. 11 is a modified example of the stent 40, which is formed of a rectangular wire or cannula 44 filled with a drug 23. The rest of the structure is the same as the fourth embodiment.

In the stent of the invention, at least one hollow portion with an open surface is formed in the body member of the stent, and the medically treating material, i.e. drug, is filled in the hollow portion. The drug can be released from the hollow portion slowly to effectively treat a portion where the stent is disposed.

While the invention has been explained with reference to the specific embodiments of the invention, the explanation is illustrative and the invention is limited only by the appended claims.

What is claimed is:

1. A stent comprising,
a body member arranged in a cylindrical shape for forming the stent to be placed in a body lumen,
at least one hollow portion with at least one open surface formed in the body member, and
a medically treating material disposed in the at least one hollow portion so that the medically treating material held in the hollow portion is gradually released through the at least one open surface to treat a portion where the stent is disposed for a desired term.

2. A stent according to claim 1, wherein said at least one hollow portion is at least one groove formed in one of inner and outer surfaces of the body member and extending along a longitudinal direction of the body member.

3. A stent according to claim 2, wherein said at least one groove is a continuous groove formed in a substantial area of the body member.

4. A stent according to claim 3, wherein said body member includes a plurality of elongated members and a plurality of connecting members situated between the elongated members, said elongated members and connecting members being arranged such that when the stent is enlarged, the elongated members are bent relative to the connecting members, and said at least one groove is a plurality of elongated grooves formed at the elongated members.

5. A stent according to claim 1, wherein said at least one hollow portion is a plurality of holes penetrating through the body member in a direction perpendicular to a longitudinal direction of the body member.

6. A stent according to claim 6, wherein said body member includes a plurality of elongated members and a plurality of connecting members situated between the elongated members, said elongated members and connecting members being arranged such that when the stent is enlarged, the elongated members are bent relative to the connecting members, said plurality of holes being formed in the elongated members.

7. A stent according to claim 1, wherein said at least one hollow portion with at least one open surface is formed of a central hole extending along and inside the body member, and a plurality of outer holes communicating between the central hole and an outside.

8. A stent according to claim 7, wherein said at least one hollow portion is a cannula forming a wire stent.

9. A stent according to claim 1, wherein said open surface has a size to gradually release the medically treating material therethrough.
